(19) United States
(12) Patent Application Publication
(10) Pub. No.: US 2026/0091200 A1
(43) Pub. Date: Apr. 2, 2026

(54) STYLET FOR SELECTIVELY OCCLUDING AN INDWELLING CATHETER

(71) Applicant: Becton, Dickinson and Company, Franklin Lakes, NJ (US)

(72) Inventors: Adam J. Boud, Bluffdale, UT (US); Haley Abramson, Costa Mesa, CA (US); Daniel Blanchard, Bountiful, UT (US); Chad Alan Tagge, Sandy, UT (US); Andrew Richards, Durham, NC (US); Brooke Treece, Salt Lake City, UT (US)

(21) Appl. No.: 19/207,093

(22) Filed: May 13, 2025

Related U.S. Application Data
(60) Provisional application No. 63/648,807, filed on May 17, 2024.

(51) Int. Cl. A61M 25/01; A61B 5/15; A61M 25/00

(52) U.S. Cl. CPC A61M 25/0102; A61B 5/150992; A61M 2025/0018

(57) ABSTRACT

A stylet may be used to perform measurements via an indwelling catheter. The stylet can be configured in various ways to selectively occlude the indwelling catheter. Because of this selective occlusion, the indwelling catheter can be used for blood draws, infusions, and other functions even while the stylet remains extended through the indwelling catheter.

### RELATED APPLICATIONS

This application claims the benefit of U.S. Provisional Patent Application No. 63/648,807, which was filed on May 17, 2024, which is incorporated herein in its entirety.

### BACKGROUND

An arterial catheter may be indwelling to provide ongoing access to a patient's blood for withdrawal, infusion, measurement, etc. To perform measurements, a device such as a stylet may be inserted into the indwelling catheter.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a stylet that may be used to perform measurements via an indwelling catheter. The stylet can be configured in various ways to selectively occlude the indwelling catheter. Because of this selective occlusion, the indwelling catheter can be used for blood draws, infusions, and other functions even while the stylet remains extended through the indwelling catheter.

In some embodiments, a stylet for use in an indwelling catheter may include a shaft, an occluding portion coupled to the shaft, and one or more sensors. The occluding portion may be configured to selectively occlude a distal tip of the catheter.

In some embodiments, the occluding portion may be configured to selectively occlude the distal tip of the catheter when the stylet is moved within the catheter.

In some embodiments, the occluding portion may extend from a distal end of the shaft and may form a distal end of the stylet.

In some embodiments, the one or more sensors may include a first sensor positioned on a distal end of the occluding portion.

In some embodiments, the one or more sensors may include at least one sensor positioned at a proximal end of the occluding portion such that the at least one sensor is isolated within the catheter when the occluding portion occludes the distal tip of the catheter.

In some embodiments, the occluding portion may be spaced proximally from a distal end of the stylet.

In some embodiments, the one or more sensors may include at least one sensor positioned on a portion of the shaft that extends distally from the occluding portion.

In some embodiments, the stylet may include a second occluding portion.

In some embodiments, the shaft may be configured to maintain a fluid pathway within the catheter when the shaft extends through the catheter.

In some embodiments, the shaft may have a circular cross-sectional shape.

In some embodiments, the shaft may have a non-circular cross-sectional shape.

In some embodiments, the stylet may include a tube that selectively blocks an opening in a sidewall of the catheter, and the occluding portion may extend distally from the tube.

In some embodiments, the occluding portion may be configured to selectively occlude the distal tip of the catheter when the occluding portion is inflated.

In some embodiments, the shaft may include a lumen having an opening that is positioned within the occluding portion. The lumen may form a pathway for inflating and deflating the occluding portion.

In some embodiments, a vascular measurement device may include a stylet that is configured to extend through a catheter. The stylet may include a shaft, an occluding portion, and one or more sensors. The occluding portion may be configured to selectively occlude a distal tip of the catheter.

In some embodiments, the occluding portion may selectively occlude the distal tip of the catheter when moved within the catheter.

In some embodiments, the occluding portion may selectively occlude the distal tip of the catheter without being moved within the catheter.

In some embodiments, the shaft may form a fluid pathway for drawing blood.

In some embodiments, the one or more sensors may include one or more of: a sensor positioned distal to the occluding portion; a sensor positioned along the occluding portion; or a sensor positioned proximal to the occluding portion.

In some embodiments, a stylet for use in an indwelling catheter may include a shaft having a distal end, an occluding portion coupled to the distal end of the shaft, and one or more sensors positioned on the occluding portion.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIGS. 1A-1C are cross-sectional side views of an example stylet in an occluded, retracted and extended position respectively within an indwelling catheter in accordance with one or more embodiments;

FIGS. 2A-2E are side views of stylets with different configurations in accordance with one or more embodiments;

FIGS. 3A-3E are cross-sectional distal views of stylets with different cross-sectional shapes in accordance with one or more embodiments;

FIGS. 4A and 4B are cross-sectional side views of an example stylet and example indwelling catheter in accordance with one or more embodiments in which a separate tube is employed to facilitate blood draw while the stylet is extended into the catheter; and

FIGS. 5A and 5B are cross-sectional side views of an example stylet within an indwelling catheter and FIG. 5C is a cross-sectional side view of the example stylet in isolation in accordance with one or more embodiments in which the stylet has an expandable outer diameter that selectively occludes the indwelling catheter.

### DESCRIPTION OF EMBODIMENTS

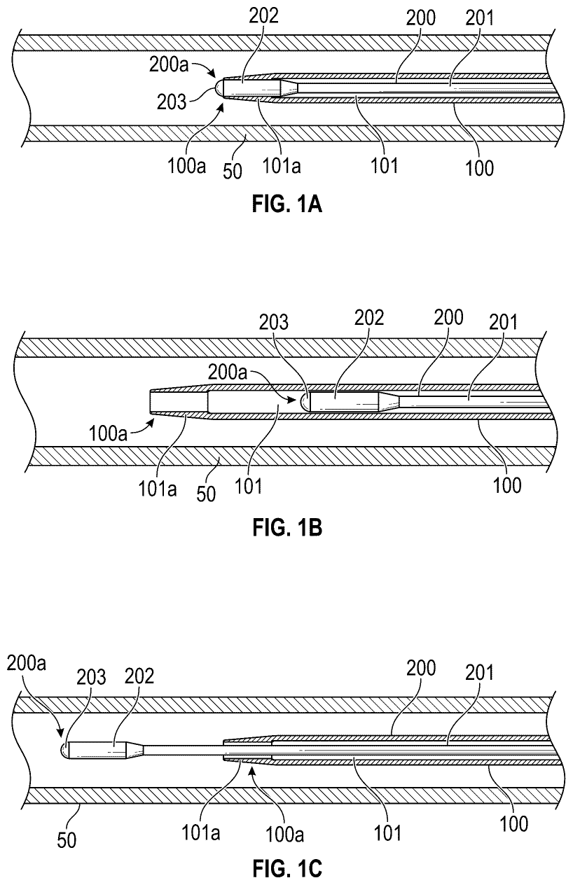

FIGS. 1A-1C are cross-sectional side views of an example stylet 200 that is extended through a catheter 100 that is indwelling within a patient's vasculature 50 in accordance with one or more embodiments. Catheter 100 has a tapered distal tip 100a that is positioned within vasculature 50 and a lumen 101 that extends through distal tip 100a to thereby form a fluid pathway for withdrawing blood from vasculature 50 and/or for infusing medicaments, saline, or other fluid into vasculature 50. A distal portion 101a of lumen 101 may have a reduced diameter relative to the diameter of proximal portions of lumen 101.

Stylet 200 includes a shaft 201 which may extend proximally to a housing/delivery device (not shown) within which the stylet's circuitry may be housed. Stylet 200 also includes an occluding portion 202 which may be positioned at, and may form part of, a distal end 200a of stylet 200. Stylet may further include one or more sensors 203 (e.g., fiber optic, piezoelectric, etc.) which may be used to collect any number of metrics (e.g., temperature, pressure, oxygen levels, etc.). In the embodiments depicted in FIGS. 1A-1C, a sensor 203 (or possibly multiple sensors 203) is positioned at distal end 200a and forms a distal tip of stylet 200.

Occluding portion 202 can have an outer diameter that is substantially the same as the diameter of distal portion 101a of lumen 101 such that occluding portion 202 occludes catheter 100 when occluding portion 202 is positioned within distal portion 101a. For example, in FIG. 1A, stylet 200 has been extended into catheter 200 until sensor 203 has passed beyond distal end 100a. With stylet 200 in this position, distal portion 101a of lumen 101 and occluding portion 202 of stylet 200 may be in sufficient contact to occlude catheter 100. However, sensor 203 can be positioned within vasculature 50, including in direct contact with blood within vasculature 50, to thereby enable sensor 203 to perform one or more measurements. In some embodiments, shaft 201 may form a conduit through which one or more communication cables extend to connect sensor 203 with the corresponding electronics.

In FIG. 1B, stylet 200 has been retracted proximally so that occluding portion 202 is proximal to distal portion 101a. The outer diameter of occluding portion 202 may be less than the diameter of the proximal portion of lumen 101 so that a fluid pathway exists around occluding portion 202 when stylet 200 is in this retracted position. Accordingly, blood draws, fluid infusion, or other flow of fluid through lumen 101 can be performed while stylet 200 remains extended through catheter 100. Also, because stylet 200 remains within catheter 100, sensor 203 could be used for one or more measurements.

In FIG. 1C, stylet 200 has been extended distally so that occluding portion 202 is fully beyond distal portion 101a. Due to the reduced diameter of shaft 201, catheter 100 is not occluded with stylet 200 in this extended position. Therefore, as with the retracted position, blood draws, fluid infusion, or other flow of fluid through lumen 101 can be performed while stylet 200 remains in catheter 100 in this extended position. Also, because it is positioned within vasculature 50 when stylet 200 is in the extended position, sensor 203 could be used for one or more measurements.

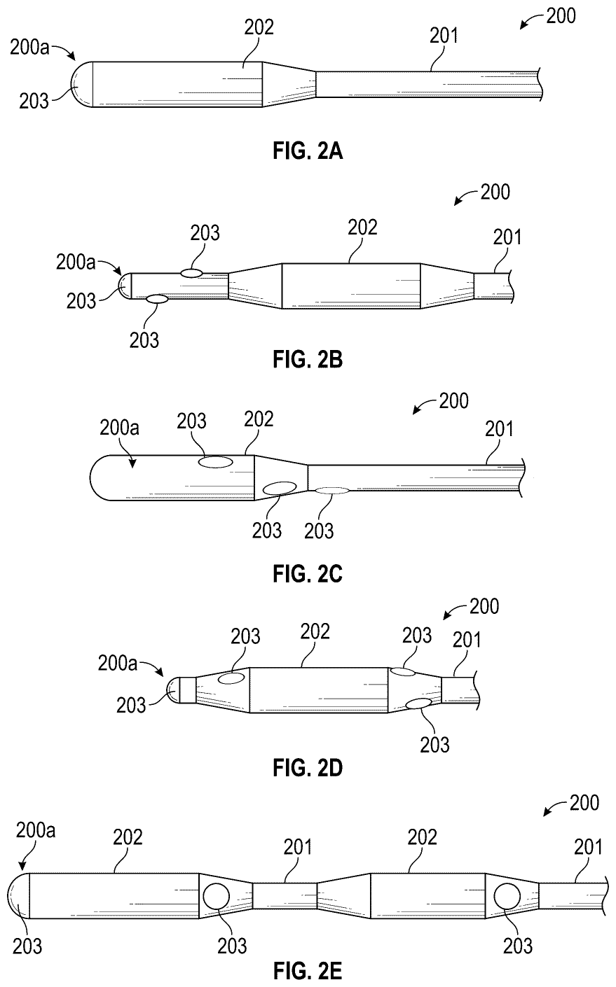

FIGS. 2A-2E each provide an example of how stylet 200 could be configured in accordance with one or more embodiments. In FIG. 2A, stylet 200 matches what is shown in FIGS. 1A-1C. One benefit of positioning sensor 203 on the distal tip of stylet 200 is that stylet 200 does not need to protrude from distal end 100a of catheter 100 to use sensor 203, and therefore trauma to vasculature 50 can be minimized. In embodiments where a sensor 203 forms the distal tip of stylet 200, a protective tube could initially extend distally beyond the sensor 203 to protect it during insertion of stylet 200. Stylet 120 could be configured to allow this protective tube to be withdrawn proximally once stylet 200 is positioned as desired within vasculature 50 or catheter 100 (e.g., by providing a mechanism at the proximal end of the protective tube that can be pulled).

In FIG. 2B, occluding portion 202 is spaced proximally from distal end 200a such that a portion of shaft 201 extends distally from occluding portion 202 to distal end 200a. Also, in FIG. 2B, two sensors 203 are positioned along this distal portion of shaft 201 in addition to sensor 203 that forms the distal tip of stylet 200. One benefit of the configuration in FIG. 2B is that multiple sensors 203 can be extended from catheter 100 while occluding portion 202 occludes catheter 100.

In FIG. 2C, stylet 200 is generally the same as in FIG. 2A except for the positioning of sensors 203. In particular, FIG. 2C represents that one or more sensors 203 could be positioned anywhere along occluding portion 202 including on its sloped proximal end 201b and that one or more sensors 203 could be positioned on shaft 201 proximal to occluding portion 202. One benefit of the configuration in FIG. 2C is that sensors 203 remain proximal to distal end 100a of catheter 100 and can therefore be isolated from blood to minimize biofouling and contamination. Then, when it is desired to use sensors 203, stylet 200 can be advanced sufficiently distally to expose sensors 203 to blood in vasculature 50.

In FIG. 2D, occluding portion 202 includes a sloped distal portion 202a and sloped distal portion 202b, both of which include sensors 203. Occluding portion 202 is spaced slightly proximally from distal end 200a such that a distal portion of shaft 201 forms distal end 200a. A sensor 203 forms the distal tip of this distal portion of shaft 201. With this configuration, the combined benefits of the configurations in FIGS. 2B and 2C can be accomplished.

In FIG. 2E, stylet 200 includes two occluding portions 202 with a length of shaft 201 extending therebetween. Sensors 203 could be positioned anywhere along either or both occluding portions 202 and along shaft 201. With this configuration, the combined benefits of the configurations in FIGS. 2A-2D can be accomplished.

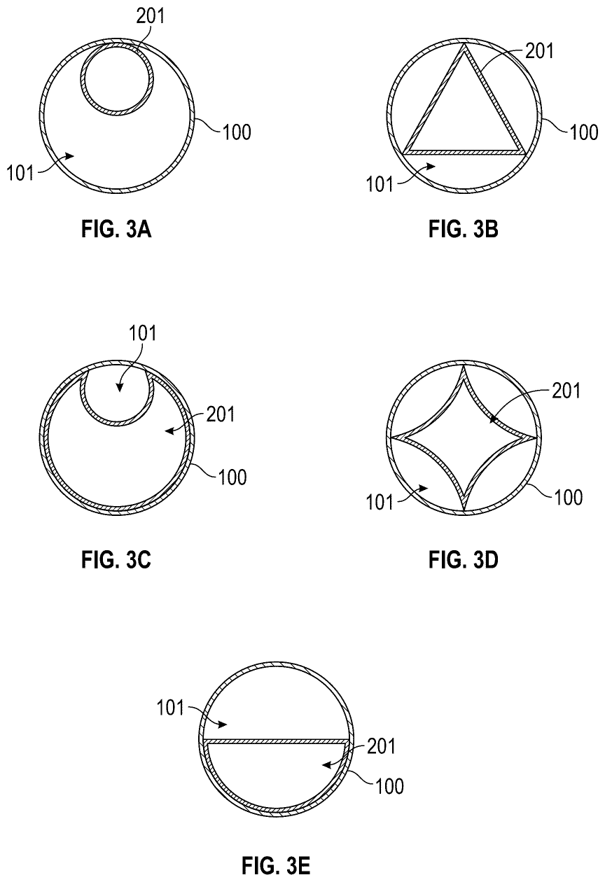

FIGS. 3A-3C are cross-sectional distal (or proximal) views of catheter 100 and stylet 200 and provide examples of suitable cross-sectional shapes of shaft 201. In each example, the shape (circular, triangular, crescent, star, and half-circular respectively) and size of shaft 201 is configured to ensure that shaft 201 does not occlude lumen 101. In some embodiments, and regardless of the cross-sectional shape of shaft 201, a fluid pathway may be formed within shaft 201 (e.g., shaft 201 may be hollow) to thereby enable blood draws through shaft 201. In such embodiments, the proximal end of shaft 201 could form a suitable connector (e.g., a female luer lock) for connecting blood draw components to shaft 201. Any communication cables within shaft 201 could be routed through this fluid pathway or through a separate pathway within shaft 201. In either case, the proximal end of shaft 201 could include a suitable interface for routing the communication cable(s) out from shaft 201 and to the corresponding circuitry.

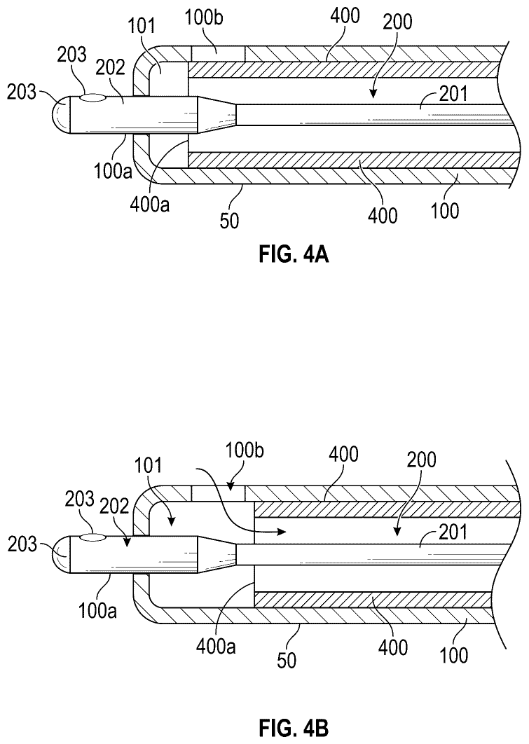

FIGS. 4A and 4B provide another example of how stylet 200 may be used within catheter 100. In this example, stylet 200 is configured in the same manner as in FIGS. 1A-1C except that a second sensor 203 is positioned along occluding portion 202. Any other suitable sensor configuration could also be used. These figures represent embodiments where a tube 400 is slidably positioned within catheter 100 and includes a distal opening 400a. To allow blood draws to be performed through tube 400, catheter 100 can include an opening 100b in its sidewall that is covered/closed when tube 400 is in a distal position as shown in FIG. 4A and uncovered/opened when tube 400 is in a proximal position as shown in FIG. 4B. Accordingly, even while occluding portion 202 of shaft 200 occludes catheter 100, blood draws may still be performed by withdrawing tube 400 within catheter 100. In some embodiments, tube 400 could be a component of stylet 200 (e.g., tube 400 and stylet 200 could be inserted together into catheter 100). In other embodiments, tube 400 could be a component of catheter 100 (e.g., catheter 100 could be supplied with tube 400 already inserted). In further embodiments, tube 400 could be a separate component from both catheter 100 and stylet 200.

Notably, in some embodiments which include tube 400, stylet 200 could include a sensor 203 positioned on or towards the proximal end of occluding portion 202 (or on shaft 201) so that the sensor remains within catheter 50 when occluding portion 202 occluded catheter 50. In this way, the proximally positioned sensor 203 will remain isolated from blood until tube 400 is withdrawn (i.e., until a blood draw is performed).

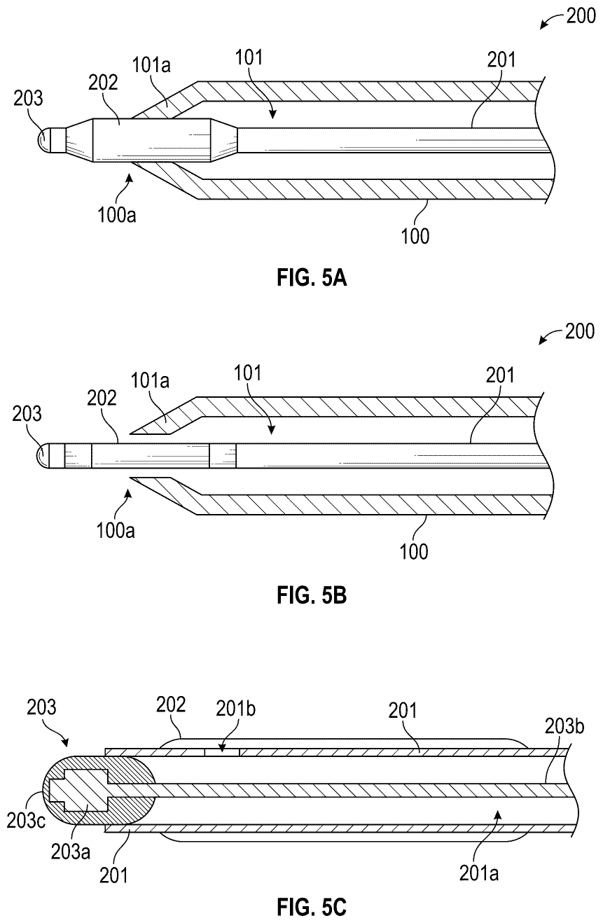

FIGS. 5A-5C provide an example of how stylet 200 could be configured in some embodiments. As represented in FIGS. 5A and 5B, occluding portion 202 is configured to selectively inflate (e.g., like a balloon) such that catheter 50 can be selectively occluded without moving stylet 200. For example, a source of air (or fluid) could be coupled to the proximal end of shaft 201 and could be used to inflate occluding portion 202 when it is desired to occlude catheter 100. Then, occluding portion 202 could be deflated when it is desired that catheter 100 is no longer occluded. This configuration could be employed in any of the above-described embodiments.

FIG. 5C provides an example of how this inflatable configuration of occluding portion 202 could be accomplished. As shown, shaft 201 includes a lumen 201a having an opening 201b that is positioned within occluding portion 202. Air can therefore flow into and out from occluding portion 202 via opening 201b. A pump or other source of air could be coupled to shaft 201 to selectively seal lumen 201a. In some embodiments, shaft 201, or at least the portion of shaft 201 extending through occluding portion 202, could be formed of polyimide.

FIG. 5C also shows that sensor 203 could include the sensing element(s) 203a that are communicatively coupled to corresponding circuitry via communication cable(s) 203b and that may be housed within an elastomeric cover 203c which forms the distal tip of stylet 200.

As can be seen, embodiments of the present disclosure enable any number of sensors to be positioned along stylet 200 distal to, proximal to, or both distal and proximal to occluding portion 202. Occluding portion 200 facilitates isolating blood from the inside of catheter 50 and can therefore minimize the risk of thrombosis and/or unintended occlusion of catheter 50.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A stylet for use in an indwelling catheter, the stylet comprising:
a shaft;
an occluding portion coupled to the shaft, the occluding portion being configured to selectively occlude a distal tip of the catheter; and
one or more sensors.

2. The stylet of claim 1, wherein the occluding portion is configured to selectively occlude the distal tip of the catheter when the stylet is moved within the catheter.

3. The stylet of claim 1, wherein the occluding portion extends from a distal end of the shaft and forms a distal end of the stylet.

4. The stylet of claim 3, wherein the one or more sensors include a first sensor positioned on a distal end of the occluding portion.

5. The stylet of claim 1, wherein the one or more sensors include at least one sensor positioned at a proximal end of the occluding portion such that the at least one sensor is isolated within the catheter when the occluding portion occludes the distal tip of the catheter.

6. The stylet of claim 1, wherein the occluding portion is spaced proximally from a distal end of the stylet.

7. The stylet of claim 6, wherein the one or more sensors include at least one sensor positioned on a portion of the shaft that extends distally from the occluding portion.

8. The stylet of claim 1, further comprising:
a second occluding portion.

9. The stylet of claim 1, wherein the shaft is configured to maintain a fluid pathway within the catheter when the shaft extends through the catheter.

10. The stylet of claim 9, wherein the shaft has a circular cross-sectional shape.

11. The stylet of claim 9, wherein the shaft has a non-circular cross-sectional shape.

12. The stylet of claim 1, further comprising:
a tube that selectively blocks an opening in a sidewall of the catheter;
wherein the occluding portion extends distally from the tube.

13. The stylet of claim 1, wherein the occluding portion is configured to selectively occlude the distal tip of the catheter when the occluding portion is inflated.

14. The stylet of claim 13, wherein the shaft includes a lumen having an opening that is positioned within the occluding portion, the lumen forming a pathway for inflating and deflating the occluding portion.

15. A vascular measurement device comprising:
a stylet that is configured to extend through a catheter, the stylet including a shaft, an occluding portion, and one or more sensors, wherein the occluding portion is configured to selectively occlude a distal tip of the catheter.

16. The vascular measurement device of claim 15, wherein the occluding portion selectively occludes the distal tip of the catheter when moved within the catheter.

17. The vascular measurement device of claim 15, wherein the occluding portion selectively occludes the distal tip of the catheter without being moved within the catheter.

18. The vascular measurement device of claim 15, wherein the shaft forms a fluid pathway for drawing blood.

19. The vascular measurement device of claim 15, wherein the one or more sensors include one or more of:
a sensor positioned distal to the occluding portion;
a sensor positioned along the occluding portion; or
a sensor positioned proximal to the occluding portion.

20. A stylet for use in an indwelling catheter, the stylet comprising:
a shaft having a distal end;
an occluding portion coupled to the distal end of the shaft; and
one or more sensors positioned on the occluding portion.
